(19) United States
(12) Patent Application Publication
(10) Pub. No.: US 2009/0022665 A1
(43) Pub. Date: Jan. 22, 2009

(54) Evaluation Of Immediate-Effect Skin Molding Agents Using DISC

(76) Inventors: Afriat Isabelle, Plainview, NY (US); Rafailovich Miriam, Plainview, NY (US)

Correspondence Address: THE ESTEE LAUDER COS, INC, 155 PINELAWN ROAD, STE 345 S, MELVILLE, NY, 11747, US

(21) Appl. No.: 11/840,622

(22) Filed: Aug. 17, 2007

Related U.S. Application Data
(60) Provisional application No. 60/823,056, filed on Aug. 21, 2006.

(51) Int. Cl. A61K 49/00; G06K 9/00

(52) U.S. Cl. 424/9.1; 382/128

(57) ABSTRACT

The present invention pertains to methods of characterizing the effects on the skin of skin-molding agents and to methods of characterizing the physical behavior of the skin-molding agents themselves. A preferred embodiment of the present invention uses a non-invasive, in vivo form of digital image speckle correlation to track deformation of human skin caused by the application of a skin-molding agent, in particular, an immediate-effect skin molding agent. The skin-molding agent may or may not be part of a composition. The application to the skin of a skin-molding agent may or may not be part of a treatment regimen. The present invention may be applied to any surface of the human body, to non-humans and to inert matter. Unlike in vitro methods and unlike invasive, in vivo methods that tension the skin with an apparatus, the present invention relies on the applied skin-molding agent to create before and after deformation images. From those images, it is possible to develop quantitative and qualitative characterizations of skin's movement and of the skin-molding agent itself. These characterizations will aid in the development of improved and/or customized skin molding agents and improved compositions comprising skin-molding agents. Generally, the data collected by the methods described herein, may also suggest improved and/or customized treatment regimens for molding the skin.

This application claims the benefit of U.S. 60/823,056, filed Aug. 21, 2006.

### FIELD OF THE INVENTION

The present invention pertains to the fields of cosmetics and dermatology, specifically to methods of characterizing the effects on the skin of skin-molding agents and to methods of characterizing the physical behavior of the skin-molding agents themselves.

### BACKGROUND

Human skin is affected by many deteriorative exogenous or endogenous factors, including gravity, topical dermatologics and makeup, pollution and sun exposure, smoking and second hand smoke, pharmaceuticals, oral supplements, diet, exercise, trauma and chronological aging. Structural changes in the skin that are associated with some of these factors, include the deterioration of the collagen and elastin network in the surface layers of the skin. This deterioration causes loss of skin elasticity and firmness, and leads to sagging and wrinkled skin. These changes in skin structure are not generally isotropic nor homogeneous in the affected area, and they are usually considered to have a negative effect on an individual's appearance. Apart from deterioration, some people are simply not satisfied with the appearance of their skin. For these reasons, numerous products have been developed, which products are intended to improve the appearance of the skin. One class of products may be known as “instant lift” products, such as “instant face lift”, “instant neck lift” or “instant chest lift” products, to name a few. These products are topically applied and cause the skin in the area of application to deform in a way that complements the appearance of the individual. “Firming” and “toning” are descriptors often used to explain the action of these products. Active ingredients, responsible for the instant or nearly instant effect, include film formers and especially polymers. Many cosmetically acceptable film formers are known. See, for example, the International Cosmetic Ingredient Dictionary and Handbook, section on Product Categories, tenth edition (herein incorporated by reference) or any recent edition. On application, the film forming product forms a continuous coating over the skin. When the product dries, the polymer remains on the skin as an invisible film that tensions the skin.

Throughout this specification, skin-molding agent refers to any agent or combination of agents that, after topical application, tensions the skin. The skin-molding agent may remain effective and the added tension may remain in the skin for a short or a substantial period. A short period may be about several seconds to several minutes while a substantial period may be, for example, one to sixteen hours or more.

Broadly, skin-molding agents can shrink or stretch the skin to which the agent is applied. Perhaps the more intuitive action is that of shrinking. Following an application of a skin molding agent, such as a polymer-containing topical composition, the solvent in the composition quickly evaporates. As it does, one or more polymers or skin-molding agents shrinks, drawing with it, the skin to which the agent has been applied. The skin to which the agent was applied is drawn in a direction toward the area of application. The shrinking may be sufficient to create a tactile sensation that is perceptible by the user. The user can feel the tensioning in his/her skin and has an immediate or almost immediate confirmation that the product is working. The shrinking is also sufficient to be measurable and, potentially, visible.

Perhaps less intuitive is that, upon drying, some compositions containing skin-molding agents expand rather than contract. As it expands, such a composition draws the skin with it, stretching the skin to which the agent was applied in a direction away from the area of application. Again, the effect can be felt and measured.

Some skin molding agents are used primarily to effect a visible change in the appearance of the user's skin. Sometimes skin-molding agents are used solely for the sensorial experience that they can deliver to the user, without creating a substantially visible change in appearance of the skin. In these cases, the composition in which the skin-molding agent resides may have within it other actives whose purpose is to firm and tone the skin relatively gradually, over a longer term such as weeks or months. In this case, the immediate-effect, skin-molding agents may be included as a device, to encourage the user to remain on the longer course of treatment. Nevertheless, even in this circumstance, users often feel gratified when they receive a tactile confirmation of the composition's potency.

Furthermore, it is important, although presently not practical, to ensure that the tensioning supplied by the skin-molding agent, not work against the firming and toning of the longer term treatment. It is a disadvantage if the immediate-effect skin molding agents tension the skin in a direction opposed to the firming and toning agents. Conversely, it would be advantageous if the immediate-effect agents could always be chosen to act in the same direction as the longer term firming and toning agents.

Presently, the applicant knows of no reliable methods for qualifying the direction of activity or quantifying the strength of the effect. Likewise, it is important that the immediate-effect tensioning does not act to worsen a user's appearance. For example, it is disadvantageous if the direction of skin-molding is such that a nearby wrinkle is made more pronounced. So again, there is a need to quantify and qualify the effect created by immediate-effect, skin-molding agents. Presently, the applicant knows of no reliable methods for so doing.

Furthermore, depending on the magnitude and direction of the tensioning, the immediate effect may be uncomfortable for the user. The stretching may be “too much”, creating irritation or adverse sensations. Here is a further need for improvement in the area of cosmetic, dermatological and pharmaceutical products that use immediate-effect skin-molding agents. This need is addressed in the present invention for the first time.

Methods for quantifying and characterizing the texture and appearance of human skin include macroscopic and microscopic techniques. Macroscopic techniques often involve a subjective assessment by a human agent. The downside to this is that there will always be a degree of uncertainty in assigning graded values to physical features based on human observation, no matter how well trained a human agent may be. To overcome this, various instrument-aided techniques have been developed that remove some or all of the human element, thus lessening the uncertainty. Techniques that rely on optical instrumentation are known and the simplest optical instrument is the camera. Photographs of test subjects are taken so that the image may be analyzed rather than analyzing the test subject directly. This has the benefit of capturing the physical features in a fixed form so they can be analyzed over an extended period of time. Measurements may be taken directly from the photograph; for example, the length of wrinkles in the skin may be accurately determined. Alternatively, the features under investigation may be identified on the photographic image and then classified within a previously defined classification scheme. The act of classification may be made by a human agent or by optical equipment, which may include optical scanning and processing software. These techniques say little or nothing about the mechanical properties of the skin itself and they are most useful only if a statistically meaningful scale has been previously defined. (See, for example, “Comparison of Age-Related Changes In Wrinkling and Sagging of the Skin In Caucasian Females and In Japanese Females”; Tsukahara et al.; Journal of Cosmetic Science; July/August 2004, vol. 55, no. 3, pp. 373-385.)

In contrast, mechanical properties of skin and other soft tissues have been investigated using various techniques common in mechanical engineering and materials testing. Many of these techniques measure the surface displacement and strain of a test sample under constant load. From these measurements, intrinsic properties such as elasticity, Young's modulus, tensile strength and hardness may be derived. These techniques have even been applied to living tissue with the aim of finding local discontinuities in the tissue. Such discontinuities may be indicative of a pathological process at work, altering the mechanical properties of the tissue. Some measurements of this type use invasive contact methods and equipment generally associated with materials testing, for example, a durometer for hardness testing, a strain gauge for tensile testing, suction cup and torsional methods for elasticity, etc. Often, it is not practical to perform these tests in vivo.

Less invasive methods of measuring mechanical properties of skin include optical methods. Recently, a non-contact, in situ technique for measuring skin stretch was reported using the optical properties of the skin and the reflection of light from the skin surface (see, “Measurement Of Skin Stretch Via Light Reflection” Guzelsu, et al.; Journal of Biomedical Optics January 2003, vol 8, 1, 81-86). The premise in that article is that as the skin is stretched, the roughness of the tissue is reduced, resulting in a smoother reflecting surface and an increase in polarized light reflected from the skin. This technique can measure the changes that takes place in light intensity due to applied skin stretch, but the measurement is only a gross average over the entire skin sample. In contrast, it is known that skin, under tension, behaves anisotropically. Because of this, measurements of the skin's response as a gross average over the whole affected area, are of limited value. Therefore, a more discrete description of the distribution of the skin displacement during stretch is of interest, especially a description that maps, in two dimensions, the surface deformation patterns resulting from skin stretching. This reference does not disclose or suggest the in vivo techniques of the present invention, nor does it disclose a method of evaluating the effects on the skin of skin-molding agents and polymers. Furthermore, methods of developing immediate-effect skin molding compositions with improved efficacy are not disclosed.

Various forms of digital image correlation have been developed, but generally, they all seek to measure the displacement and deformation gradients caused by a load applied to a surface. They do this by correlating small regions of a digital image made after deformation with those same regions on a digital image made before deformation. When this correlation is carried out at many points over the whole image of the specimen under investigation, it yields a vector displacement field for the deformed surface. From this displacement field, stress, strain and Young's modulus may be computed.

One digital image correlation technique, in particular, is digital image speckle correlation. Digital image speckle correlation (DISC) has been in use and development for more than two decades to analyze the response of materials to stress and the environment. In principle, all types of materials, living and non-living, may be studied with DISC. Generally, geometric features are identified in the field of a digital image before deformation and then these features are tracked to their new location in the image field after deformation. By this tracking, a vector displacement field for the deformed surface can be constructed. In the conventional method of DISC, reflective materials (speckles) are randomly distributed on the surface under examination. The speckles provide easy-to-track geometric features on the surface of the test specimen. After capturing one digital image of the undeformed surface and one digital image of the deformed surface, the images are divided into subsets. The subsets on the image of the undeformed surface are matched to the corresponding subsets on the image of the deformed surface. This is done through sophisticated numerical computer analysis, comparing patterns of light intensity in the before and after photos. The coordinates of the center points of each pair of subsets define a displacement vector which describes the average displacement of the subset as a result of the deformation. The displacement vectors can be resolved into vertical and horizontal components and that information may be represented as vertical and horizontal projection maps. Using numerical differentiation, the normal strain along either direction may be obtained.

In “Determining Mechanical Properties of Rat Skin With Digital Image Speckle Correlation” (Guan, et al., Dermatology, vol 208, no. 2, 2004, p. 112-119), the contents of which are herein incorporated by reference, there is described an in vitro application of DISC on samples of rat skin. Three sections of skin were tested, freshly excised skin, skin allowed to rest 24 hours after being excised and skin pre-treated for 24 hours with a commercially available cosmetic anti-wrinkle moisturizer. The skin sections were stretched in a tensile testing machine at a constant rate of 0.508 mm per minute. The speckle material consisted of 24 μm silicon carbide and talc material, which provide a high contrast black and white surface. Digital images were taken with a Kodak MegaPlus 1.6i charged-coupled device camera, having a resolution 2,029×2,048 pixels. For each skin sample, the tensile stress, tensile strain, ultimate strain, Young's modulus and break strength were determined. The article concludes, in part, that the moisturizer efficiently slowed down the loss of elasticity in the rat skin. The article further suggests, but does not describe, the use of DISC, in vivo, to monitor changes in skin elasticity, which may provide a means of predicting wrinkle formation. The article merely mentions, but does not describe, that the skin may be put under stress using a gas loading electrodynamometer. The article also suggests, but does not elaborate, that cosmetic efficacy may be measured by an in vivo DISC technique, where DISC measurements are made before and after the skin is treated with an anti-aging product. This reference does not disclose or suggest the in vivo techniques of the present invention, nor does it disclose a method of evaluating the effects on the skin of skin-molding agents and polymers. Furthermore, methods of developing immediate-effect skin molding compositions with improved efficacy are not disclosed.

A modified DISC technique has been successfully applied in vivo, using the pores of the skin for tracking deformation, rather than speckle material. (See, “Dynamic Facial Recognition With DISC: Identify the Enemies”, paper presented at the meeting of the American Physical Society, Mar. 22-26, 2004, Montreal). The musculature under the skin of the face provided the deformation of the skin and this reference describes a successful facial recognition method. The reference mentions that the technique may be used for early detection of skin disorders or skin abnormalities, but no further disclosure is made. This reference does not disclose or suggest the in vivo techniques of the present invention, nor does it disclose a method of evaluating the effects on the skin of skin-molding agents and polymers. Furthermore, methods of developing immediate-effect skin molding compositions with improved efficacy are not disclosed.

In “Investigations of Facial Recognition and Mechanical Properties of Aging Skin Through Digital Image Speckle Correlation” (submitted to the Intel Science Talent Search, November, 2004) there is disclosed an in vivo application of DISC technology to human facial skin. It was determined that age-related changes in the skin (for example, loss of elasticity) can be observed by Examining a cross section of a vector displacement map. The map is created from vector displacement data obtained in a DISC-like procedure.

None of the foregoing discloses the use of a non-invasive, in vivo, DISC-type data collection system, to quantify, qualify or otherwise evaluate the effects on the skin of skin-molding agents and polymers. Furthermore, methods of developing immediate-effect skin molding compositions with improved efficacy are not disclosed.

### OBJECTS OF THE INVENTION

A main object of the present invention is to provide a non-invasive, in vivo method of characterizing the behavior of human skin after the application of an immediate-effect skin-molding agent or composition containing an immediate-effect skin-molding agent.

Another object of the present invention is to provide a method of quantifying the strength and qualifying the direction of the effect produced by immediate-effect skin molding agents and compositions containing one or more immediate-effect skin-molding agents.

Another object is to provide methods of developing immediate-effect skin molding compositions with improved efficacy.

Another object is to provide methods of developing immediate-effect skin molding compositions with improved comfort.

Another object is to provide methods of characterizing the behavior of immediate-effect skin molding agents, so that predictive comparisons between different skin-molding agents are possible.

### DESCRIPTION OF THE FIGURES

FIG. 1 is a schematic representation of a digital image speckle correlation system used in the present invention.

FIG. 2 is an example of a horizontal projection map.

FIG. 3 is a schematic depiction of a skin sample before and after an applied strain.

FIG. 4 is an example of a vector displacement map.

FIGS. 5a and 5b are, respectively, the horizontal and vertical projections of the map of FIG. 4.

FIGS. 6a and 6b are, respectively, a minute-by-minute sequence of horizontal and vertical displacement maps, obtained during the drying of a film-forming polymer.

### SUMMARY OF THE INVENTION

A preferred embodiment of the present invention uses a non-invasive, in vivo form of digital image speckle correlation to track deformation of human skin caused by the application of a skin-molding agent, in particular, an immediate-effect skin molding agent. Throughout the specification, the term “immediate-effect” shall consistently mean that when a topical agent is applied to the skin of a living human, the agent causes a measurable, quantifiable change and/or reaction in the skin; the change is perceptible by the one to whom the agent is applied and the change occurs within less than about five minutes, preferably less than three minutes, more preferably less than one minute and, most preferably, within about ten seconds of a single application of the topical agent. The term “immediate effect” is intended to distinguish over treatment regimens that require more than about five minutes and/or multiple applications before an effect can be quantifiably measured or perceived by the user. It should also be borne in mind that the applied composition comprising an immediate-effect skin-molding agent may require longer than five minutes to completely dry on the skin, but the immediate effect will be present even while some portions of the compositions continue to dry. The skin-molding agent may or may not be part of a composition. The application to the skin of a skin-molding agent may or may not be part of a treatment regimen. The present invention may be applied to any part of the body and to non-humans. In fact, the methods described herein may be applied to inert matter, as long as that matter can be deformed by a topically applied agent. Thus, the techniques may be used in vitro, as well. But, unlike other in vitro methods and unlike invasive, in vivo methods that tension the skin with an apparatus, the present invention relies on the applied skin-molding agent to create before and after deformation images. From those images, it is possible to develop quantitative and qualitative characterizations of skin's movement and of the skin-molding agent. These characterizations will aid in the development of improved and/or customized skin molding agents and improved compositions comprising skin-molding agents. Generally, the data collected by the methods described herein, may also suggest improved and/or customized treatment regimens for molding the skin.

### DETAILED DESCRIPTION OF THE INVENTION

The present invention is generally concerned with quantitatively and qualitatively characterizing the activity of immediate-effect skin-molding agents. Immediate-effect skin-molding agents are herein distinguished from treatments requiring a longer term to manifest. The distinction is valid because in a first period of time, following the application of a composition that comprises an immediate-effect skin-molding agent, the tension in the skin and the molding of the skin is dominated by the immediate-effect skin-molding agent and any actives that manifest an efficacy after a longer term are not generally important in that first period of time. Also, studying the behavior of materials that create a measurable effect within the first several seconds and minutes of application is useful because it may be predictive of the course of a longer term treatment. For example, the present invention will be helpful in answering the following question. Is the immediate-effect additive or does it hinder the long term activity of a course of treatment?

Regarding the application to the skin, of an externally applied load, the DISC technique of the present invention is much simpler than other DISC techniques. Generally, it may not even be practical to apply such loads, in vivo. In contrast, the DISC technique of the present invention is in vivo, while being completely non invasive. Furthermore, an externally applied load would deform the skin in a manner that is different from the desired deformation achieved by a skin-molding agent. The response of the skin to an external load may bear little or no resemblance to the response that the skin undergoes due to the application of a skin-molding agent, such as a skin-molding polymer. So while a DISC technique that uses an externally applied load may be useful for measuring some physical parameters of the skin, like, Young's modulus, such a technique misses the opportunity to characterize the behavior of the skin itself, in a real life situation. The response of the skin to the application of a skin-molding polymer or other agent is characteristic of individuals. Therefore, measuring a mechanical property like Young's modulus or some other material parameter, does not give one the ability to predict the behavior of the skin nor the skin's immediate response to treatment, because the system is far too complicated and specific to each individual. Furthermore, mechanical properties like Young's modulus quantify an average behavior of materials over a period of time that is sufficient for transient behavior to subside. Therefore, such gross or average parameters may be unsuitable for predicting behavior within seconds or minutes of an applied load. In contrast, techniques of the present invention directly measure the immediate response of an individual's skin to a skin-molding polymer. Therefore, the techniques of the present invention not only avoid the complexity of relating the skin's mechanical properties to its dynamic response, but the techniques of the present invention incorporate the immediate response of an individual's skin into quantitative and qualitative characterizations of skin. This is a great advantage because the skin's response is part of what creates the individual's appearance to the rest of the world.

Likewise, the method of the present invention is different from a method described in applicants' co-pending application U.S. Ser. No. 11/296,236, herein incorporated by reference. In the '236 reference, the musculature, of an individual under evaluation, provided the displacement of the skin in before and after images. The stimulus was supplied voluntarily by the test subject. In the present invention, the skin is not displaced by a voluntary impulse supplied by the test subject. Rather it is supplied by a natural, involuntary response of the skin to one or more topically applied skin-molding agents or to a composition comprising such an agent.

Throughout this specification, the terms “comprise,” “comprises,” “comprising” and the like, shall consistently mean that a collection of objects is not limited to those objects specifically recited.

FIG. 1 is a schematic representation of a digital image speckle correlation system used in the present invention. A camera (1) for capturing digital images is a charge-coupled device providing a minimum of four mega pixel resolution. This resolution is sufficient to resolve the pores of human skin, which are the points being tracked in the technique of the present invention. Technically, virtually any feature in the image field may be useful for tracking between images, however, the success of a DISC-type technique depends on having a plethora of features to track. In humans, skin pores fulfill this requirement. Some useful cameras are Canon EOS Rebel Digital camera (6.3 mega pixel resolution), the Toshiba DK-120F CCD camera, the five-mega pixel Canon D60 or Canon Powershot Pro1. Data collected by the camera is pre-processed by a frame grabber (2), such as PIXCI® from EPIX®, and the digitized information is downloaded to a computer (3) for numerical analysis. Many research groups have developed their own software on the DISC technique to suit their own needs. Persons of ordinary skill in the art are capable of developing such software without undue burden. Furthermore, there are also commercially available software applications, one being VIC-2D from Correlated Solutions Inc., (West Colombia, S.C.), with an advertised displacement accuracy of better than one one-hundredth of a pixel. Another supplier of digital image correlation systems and software is Optical Metrology Innovations, Cork, Ireland.

A typical procedure comprises capturing at least two images. A procedure with just two images is described, and easily extended to more than two images. A first image of a surface (i.e. a portion of skin) is made before the application of an immediate effect skin-molding agent. A skin-molding agent, such as a cosmetic composition containing a skin-molding polymer, is applied to the imaged area and the skin is allowed to deform in response to the applied agent. The methods of the present invention require that the applied composition does not occlude the pores of the skin beyond the ability of the imaging equipment to resolve the pores. The skin-molding agent or composition containing the same, may initially occlude the pores, as long as at some later time (i.e. after drying) the composition does not occlude the pores. Within the time that the skin-molding agent remains effective, while the skin is in a deformed state, a second image is made. Throughout the specification, “deform” means that the skin has assumed a shape that is different from an initial shape or that the pores in the skin have assumed an arrangement that is different from an initial arrangement. Preferably, the surface to be imaged is held motionless during image capture. A harness designed to hold motionless, the part of the body containing the area of study, may be used. For example, if the area of study is on the face, a chin rest or a full head harness may be used to hold a subject's head still. Useful devices of this type are available from Canfield Scientific. It is also preferable that the camera be held immovable during picture taking. A camera stand may be used for this purpose.

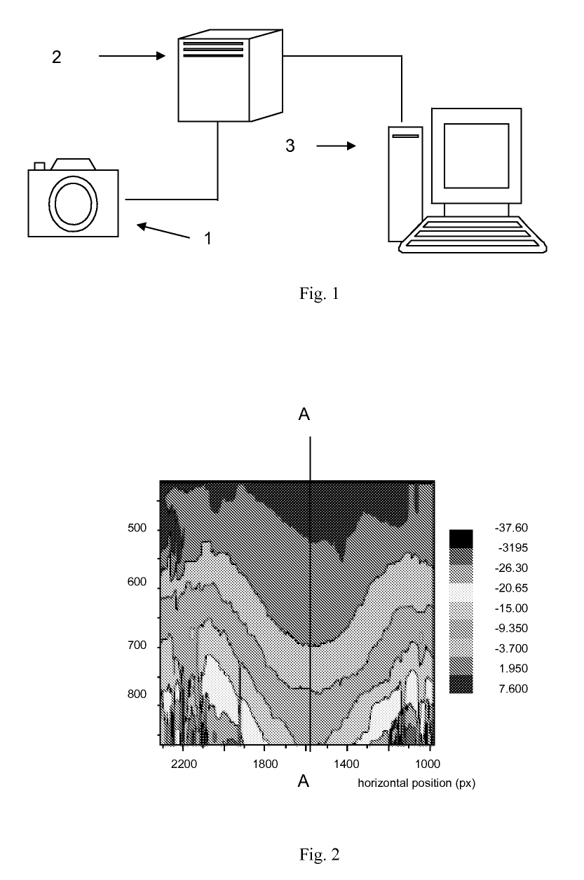

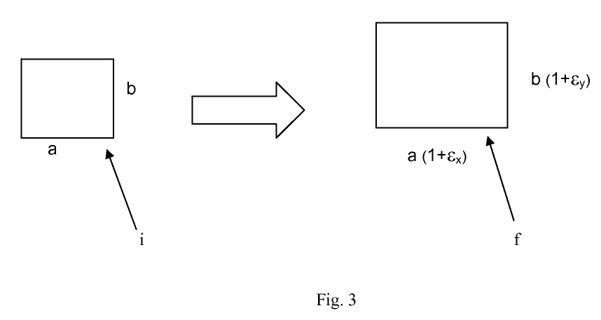

Once the images are acquired, software, such as Photoshop© from Adobe®, is useful for imposing on the images, a boundary of the area to be studied and a reference coordinate system, as well as for obtaining a rough estimate of pore displacement. The boundaries are somewhat arbitrary and may be chosen to define a domain large enough for analyzing several areas of the skin. More sophisticated image analysis software is commercially available. For example, products sold under the OriginLab® label are able to analyze DISC-type digital image data to calculate values for a host of mechanical properties of the material under investigation. The image analysis software determines the coordinates of each pore in the displacement field relative to the reference coordinate system, for the before and after image. From this data, correlations are established between the pores in the before and after images and a field of displacement vectors, as discussed above, may be generated. Each displacement vector represents the movement of one pore from its initial to final location. Each pore vector in the field of displacement vectors is resolved into its vertical and horizontal projections, from which vertical and/or horizontal projection maps may be produced. An example of a horizontal projection map is shown in FIG. 2. In the projection maps, the horizontal and vertical axes convey the coordinates of any position in the field of study. In FIG. 2, the units are pixels. Areas of constant displacement are color coded in these figures. By measuring the displacement of features (pores) at the boundary of the image area, it is, in principle, straightforward to calculate the change in size of the image area. The change in size along the vertical and horizontal directions may be separately calculated, or a total deformation of the image region may be calculated. These may be expressed as percent change in size or strain, induced in the bounded area. A simplified representation of this is shown in FIG. 3 and the equations that follow. Referring to FIG. 3, a section of skin (i) before treatment, is shown, having dimensions a and b. Thus, initially, the surface area of this skin sample is S=ab. The same section of skin after treatment with a skin-molding agent is also shown (f), having dimensions a(1+εₓ) and a(1+εy). The surface area after treatment is, thus:

S′=[a(1+εₓ)][b(1+ε_{y})]=ab(1+εₓ+ε_{y}+εxxε_{y})≅ab(1+εₓ+ε_{y}).

From this the total strain, ε, is easily seen to be:

ε=(S′−S)/S≅ab(εₓ+ε_{y})/ab=εₓ+ε_{y}

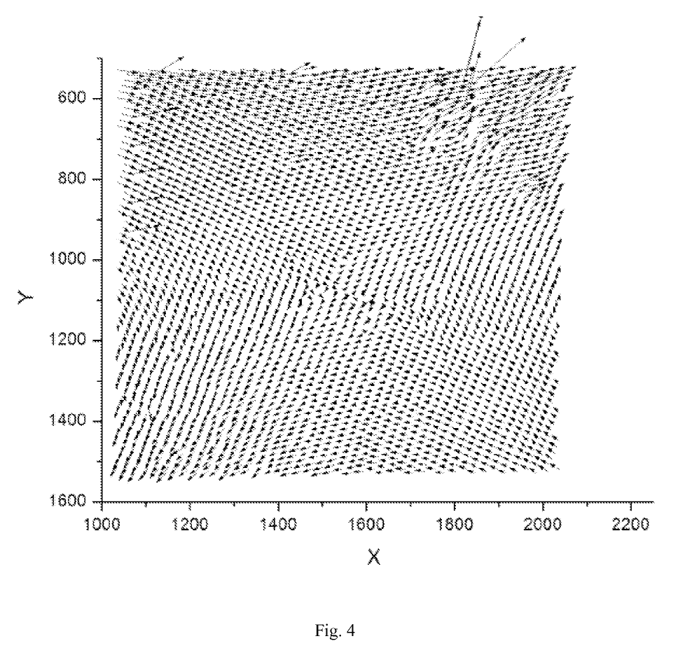

Alternatively, sufficiently sophisticated digital analysis software is available (i.e. OrignLab®) that can analyze the entire displacement field and directly calculate strain at any position and average strain over the field. With such software, a literal projection map, like that of FIG. 2, and a literal vector displacement map, like that of FIG. 4 may not be produced, but the underlying data manipulation is the same, in principle. Thus, throughout the specification, references to “projection map” and “vector displacement map” include the underlying, raw data from which such maps may be produced.

In general, total strain, ε, may be positive or negative. If ε is positive, then the skin sample may be considered to have stretched and if ε is negative, then the skin sample may be considered to have shrunk. In fact, depending on the skin-molding agent, skin may stretch or shrink. Surprisingly, the use of one or more projection maps has proven very useful in quantifying the magnitude (total strain) and qualifying the direction (positive or negative strain) of the skin's response to skin molding agents. The following non-limiting examples may increase the reader's appreciation of the present invention.

### EXAMPLE 1

### Skin Stretch/Shrink and Material Behavior

In this example, we focus on an area of the forearm. During the procedures, a device was used to hold the forearm in a fixed position. The experimental setup was first calibrated by running trials on untreated skin. Skin-molding, film-forming polymers were uniformly applied on a flat framed region (typically, 4×4 cm or 3×3 cm) of the forearm.

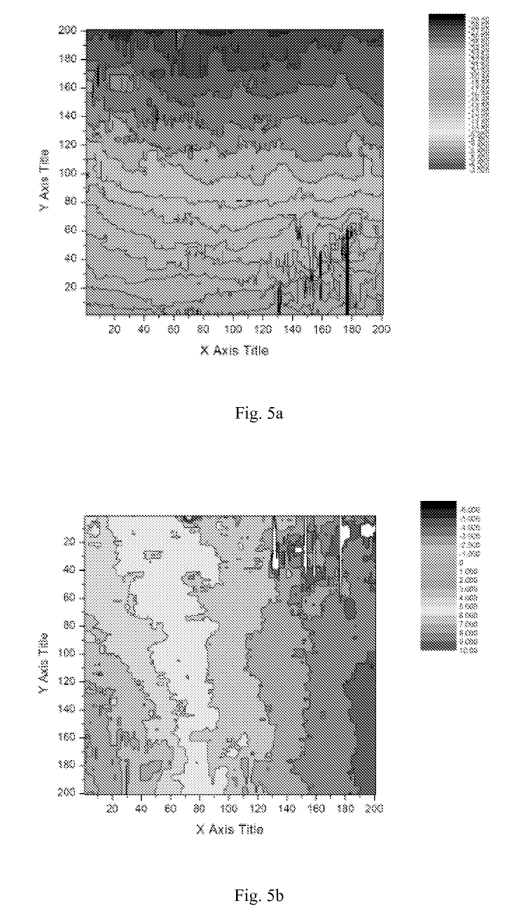

Images were acquired at defined intervals of the drying process (i.e. one minute intervals over a five to seven minute period). Experiments may be repeated to ensure accuracy. The acquired images were aligned using Mirror software (Canfield) to enable the center of the sample to be stationary and to eliminate the rotational and lateral movements of the forearm that may have occurred during the experiment. The DISC analysis was then performed on the aligned images. DISC correlates the images of the undeformed and deformed skin, subset by subset and generates a vector displacement map (see FIG. 4) and horizontal and vertical projections, thereof (see FIGS. 5a and 5b, respectively).

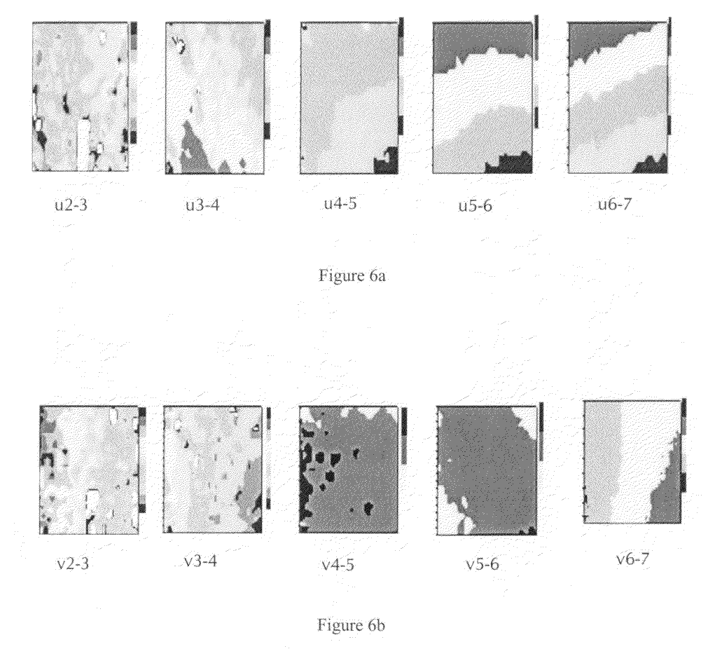

Material Behavior—The distribution of the skin deformation depends on the uniformity of the application and on the wetting properties of the skin-molding polymer. The film homogeneity and drying time can be analyzed by observation of the displacement maps during the drying process. By observing the minute-by-minute horizontal and vertical displacement maps (see FIGS. 6a and 6b, respectively), one can see the progressive changes that occur during the drying process. At the beginning of the process, the repartition of the film is not homogeneous; as the solvent evaporates, the projection maps become more homogeneous showing that the film is actually forming on the skin. The drying time corresponds to the time point at which no or insignificant additional strain is detected in the displacement maps. Thus, the techniques of the represent invention can be used to investigate the wetting-dewetting behavior of topically applied skin-deforming solutions. This, by itself, is a valuable tool for formulators. Quite unexpectedly, when the techniques of the present invention are used to produce vector displacement maps of skin pores, those maps have been found to be useful as a means of quantifying and qualifying the homogeneity, drying time and film forming process of topically applied skin-molding agents. By examining vector displacement maps, comparisons of skin-molding agents are possible, in the areas of uniformity of effect and drying time. A formulator is thus enabled to discriminate on the basis of objective data rather than subjective feedback.

Skin Stretch/Shrink—The orientation of deformation may easily be seen in the vector displacement map. By observing the vector displacement field generated by DISC, one can visualize the direction of the stretch and or shrink at any given point of the sample. The orientation of the stretch is visualized in the vector maps and the strain is calculated along the principal axis of stretch. For example, one can see in FIG. 4, that the principal directions of deformation are along the diagonals of the square. Stretch is observed along the diagonal from lower left to upper right (arrows point out). Shrink is observed along the diagonal from upper left to lower right (arrows point in). In the trial of FIG. 4, the skin-molding agent was polyvinyl pyrrolidone 5% in water (Luviskol K90, BASF). The values of the strains for the PVP were 1.3% in the stretching direction and −0.5% in the shrinking direction.

Other skin-molding agents produce different characteristic patterns, with more or less stretch and/or shrink, but in general, a principal direction of stretch and/or shrink can always be identified. The strain for untreated skin (calibration trials) and the strain for treated skin were calculated based on the formula above along the principal axes of stretch/shrink. The magnitude and characterization of stretch or shrink, some for multiple trials, are shown in table 1.

TABLE 1

| | skin | skin | sample size of |
|---|---|---|---|
| | deformation | sample | skin molding |
| skin molding agents | (%) | size (cm²) | agent (g) |
| untreated (bare) skin | 0.017 +/− 0.007 | 9 | 0 |
| hyaluronic acid 0.5% | +0.1, +0.3, +0.15 | 9 | 0.084 |
| solution | | | |
| Polylift ® (almond | −0.81, −0.86, −0.89 | 9 | 0.084 |
| protein 11% in | | | |
| water from Silab) | | | |
| Silk Crystal gel | −2.1, −2.4 | 16 | 0.14 |
| (7% in water) | | | |
| (silk protein) | | | |
| Silk 561 (7%) | −1.96, −1.93 | 9 | 0.04 |
| (silk protein) | | | |
| Avalure ® UR 450 | −2.1 | 9 | 0.04 |
| (polyurethane | | | |
| dispersion 38% in | | | |
| water from Noveon) | | | |
| Avalure ® AC 122 | +0.28 | 9 | 0.04 |
| (acrylates copolymer | | | |
| 29% in water from | | | |
| Noveon) | | | |
| Daitosol ® 5000SJ | −0.83 | 9 | 0.03 |
| (acrylate/ | | | |
| ethylhexylacrylate | | | |
| crosspolymer 50% in | | | |
| water from Kobo) | | | |

+stretching, −shrinking

Thus, the technique of the present invention quantifies and qualifies the response of the skin to the skin-molding agent. The strain measurements in untreated skin were orders of magnitude lower than treated skin, indicating that the technique of the present invention is able to filter out background noise, systematic and/or random errors. This example demonstrates that a Digital Image Speckle Correlation technique of the present invention, requiring a simple experimental setup, can map, in two dimensions, the deformation patterns induced in a sample of skin, during the drying process of a skin-molding agent. From these patterns, the distribution of the displacement at any point in the skin sample can be derived and the strain in the direction of the deformation can be calculated, thus quantifying the effect of the particular skin-molding agent, as heretofore, unknown. The technique of the present invention can be used to select and screen skin-molding agents for incorporation into cosmetic, dermatologic and pharmaceutical compositions.

### EXAMPLE 2

### Skin Stiffness

A modified technique according to the present invention is now described. This experiment focused on the back on the hand. The skin was deformed laterally using a very small probe (from Gas Bearing Electrodynamometer (GBE)) attached to the skin at the center of a defined square (1 cm 2). The skin surface contacted by the probe was a few square millimeters. The probe is capable of moving with a lateral, reciprocating action. Photographic images were acquired before and during deformation of skin, treated with (Polylift® from Rita Corporation). Non-treated skin was tested, as a control. The experiment was repeated three times for untreated skin and for skin with polymer.

Using DISC as described herein, the distribution of the skin displacement along a vertical axis was acquired and the value of maximum skin displacement can be correlated to skin stiffness. From one skin-deforming agent to another, the resulting skin stiffness can be compared by comparing the values of maximum skin displacement. By comparing the maximum amplitude of the displacement for untreated skin and for skin treated with polymer, it was observed that Polylift® (almond extract solution) stiffens the skin by a significant factor of 23%.

Thus, it is demonstrated that when coupled with a device that can deform the skin (i.e. a probe), the modified DISC technique of the present invention can measure the effect of a skin-molding agent on skin stiffness. One practical interpretation of this relative skin stiffness is as an indicator of the discomfort induced by a topically applied skin-molding agent. Another is as an indicator of the discomfort alleviated by a topically applied composition. Thus, by the simple DISC procedure just described, a formulator can determine in advance, which skin-molding agents are more likely to cause discomfort to the wearer. A formulator can also know in advance which topical treatment is likely to alleviate discomfort associated with stretched skin, as during wound healing, for example. The techniques of the present invention, therefore, find additional practical applications in the cosmetic and medical fields.

### EXAMPLE 3

### Compatibility of Skin Molding Agent and Cosmetic Foundations

The following demonstrates the technique of present invention as a tool for qualifying the interaction between a skin molding agent and a cosmetic foundation and answers the question, “Is it better to apply the foundation above or below the skin molding agent?” The skin molding agent in this test was the polymer blend shown in table 2.

TABLE 2
Skin Molding Formula

| Polymer Blend Skin Molding Formula | Percent |
|---|---|
| disodium edta | 0.120 |
| potassium sorbate | 0.150 |
| water/hydrolyzed keratin | 2.400 |
| PVP | 4.000 |
| butylene glycol/ammonium acrylates copolymer/ | 4.000 |
| C11-C15 pareth-7/sodium laureth-12 sulfate | |
| algin | 0.400 |
| glycerine | 0.500 |
| acrylates copolymer | 4.000 |
| Phenoxyethanol | 0.900 |
| citric acid | 0.075 |
| methylmethacrylate crosspolymer | 0.500 |
| polyaminopropyl biguanidine | 0.100 |
| ammonium acrylodimethyltaurate/VP copolymer | 0.750 |
| water | 82.105 |

### Experimental Procedure:

All experiments were performed in vivo on a 2×2 cm surface of the forearm. The forearm was maintained stationary as much as possible during image acquisition. To reduce the effects of involuntary movement of the forearm, the acquired images were aligned using Mirror Alignment Software from Canfield, by matching features that are outside of the area of analysis.

Compatibility of the polymer blend with each of the five foundations was investigated two ways: 1. applying the skin molding agent on top of the foundation, 2. applying the skin molding agent under the foundation. In all trials, a moisturizer was first applied, as such is typical of a beauty treatment routine. Moisturizer alone and moisturizer followed by polymer blend were used as controls. Following application of topical products, pictures were acquired every 30 seconds for a total of four minutes. Thus, the following twelve trials were run:

Order of Products applied

Moisturizer (M)
M then Polymer blend then foundation 1
M then Polymer blend then foundation 2
M then Polymer blend then foundation 3
M then Polymer blend then foundation 4
M then Polymer blend then foundation 5
M then Polymer Blend
M then foundation 1 then Polymer blend
M then foundation 2 then Polymer blend
M then foundation 3 then Polymer blend
M then foundation 4 then Polymer blend
M then foundation 5 then Polymer blend

Three of the foundations were water in silicone emulsions, one was a oil in water emulsion and one was a silicone in water emulsion.

### Results:

The compatibility of the polymer blend with each of the five cosmetic foundations was evaluated for film uniformity, skin stretch and drying time.

Moisturizer Control—The average skin displacement measured after application of moisturizer was 1.7+/−0.5 pixels, which is small and validates the moisturizer as a suitable control.

TABLE 3
Results of Skin Molding polymer blend applied below foundation

| | Moisturizer | Moisturizer | Moisturizer | Moisturizer | Moisturizer |
|---|---|---|---|---|---|
| | then Polymer | then Polymer | then Polymer | then Polymer | then Polymer |
| Formulas | blend then | blend then | blend then | blend then | blend then |
| applied in | foundation 1 | foundation 2 | foundation 3 | foundation 4 | foundation 5 |
| order | (W/Si) | (W/Si) | (W/Si) | (O/W) | (Si/W) |
| Film | Granulous | Not uniform | Not Uniform | Not uniform | Uniform |
| quality | | | | | |

TABLE 4
Results of Skin Molding polymer blend applied on top of foundation

| | | Moisturizer then | Moisturizer then | Moisturizer then | Moisturizer then | Moisturizer then |
|---|---|---|---|---|---|---|
| Formulas | Moisturizer | foundation | foundation | foundation | foundation | foundation |
| applied in | then Polymer | 1 (W/Si) then | 2 (W/Si) then | 3 (W/Si) then | 4 (O/W) then | 5 (Si/W) then |
| order | blend | Polymer blend | Polymer blend | Polymer blend | Polymer blend | Polymer blend |
| Film | Uniform | Uniform | Uniform | Uniform | Uniform | Cracking |
| quality | | | | | | |
| Drying | 2-3 min | 2-3 min | 2-3 min | 2-3 min | 2-3 min | 2-3 min |
| time | | | | | | |
| Skin | 1.3 +/− 0.3% | 0.6 +/− 0.17% | 1.2 +/− 0.35% | 1.9 +/− 0.8% | 1.0 +/− 0.2% | 0.6 +/− 0.2% |
| stretch | | | | | | |

When applied on top of the foundation, the polymer blend forms a uniform film, dries within 3 minutes and maintains part of its tightening properties on the water-in-silicone and oil-in-water foundations. When applied over the silicone-in-water foundation, the film cracks, resulting in an unacceptable appearance.

In contrast, when applied underneath the foundations, acceptable results were achieved with the silicone-in-water foundation, but not with the water-in-silicone and oil-in-water foundations, which do not dry uniformly, causing an unacceptable appearance.

Thus, the techniques of the present invention suggest, as a general rule, to apply the skin molding polymer blend on top of water-in-silicone and oil-in-water foundations, but underneath silicone-in-water foundations.

Many of the skin-molding agents with which the present invention will find use are cosmetic film forming polymers. Film formation is a complex process that depends on various factors including, but not limited to, the solvent in which the polymer is dispersed, the polymer concentration, the mechanical, chemical properties of the substrate onto which the polymer is deployed, the surface energetics of the substrate, etc. Until now, the complexity of the film forming process has made it very difficult for a formulator to choose a skin-molding polymer on the basis of quantifiable parameters of various film-forming materials. Mostly, all one could do was to formulate with one or more skin-molding materials and collect highly subjective data in clinical trials. Direct comparisons between materials based on quantifiable data, were all but impossible. In contrast, the present invention permits the collection of quantifiable data and the compilation of a database of skin-molding agents, thus enabling the optimum selection of materials for a particular application. Parameters that may be optimized by the present invention include stretch or shrink along a given direction and change in comfort level, and wetting-dewetting properties of a topical composition.

In principle, any topically applied skin-molding agent can be quantified and qualified by the techniques of the present invention. Of great interest in cosmetic formulation are film-forming polymers. Many skin-deforming polymers are water soluble and work best when incorporated into water or hydro-alcoholic base compositions. Generally, the inclusion of excessive amounts of oil is to be avoided as too much oil may make the skin more pliable and therefore lessen the stretching-shrinking effect and the sensorial experience. However, as long as some skin deformation is achieved by a composition when topically applied, the present invention is useful for quantifying and qualifying the composition in a unique fashion. Thus, there is little or no restriction on the types of compositions that may be analyzed with techniques of the present invention.

The application to the skin of a skin-molding agent may or may not be part of a treatment regimen. The present invention may be applied to any part of the body. This is beneficial because it enables direct measurement of an effect on the intended area of application, rather than having to attempt to extrapolate or guess what the effect will be. Therefore, the exact effect on the body, be it the face, neck, chest, arms, legs, etc. can be measured. Generally, the data collected by the methods described herein, may also suggest improved and/or customized treatment regimens for molding the skin. For example, different parts of the body may generally require different skin-molding agents or different concentrations of a skin-molding agent. With the present invention, the required strength of the skin-molding effect can be determined and a suitable skin-molding agent at a suitable concentration can be identified.

We claim:

1. A non-invasive, in-vivo method of characterizing the effects, on the skin, of a skin-molding agent, comprising the steps of:
applying to a portion of skin, a skin-molding agent or a composition comprising a skin-molding agent; and
tracking the displacement of skin pores caused by the application of the skin-molding agent.

2. The method of claim 1 wherein the characterization includes quantifying the strength and qualifying the direction of the effect produced by one or more immediate-effect skin molding agents.

3. The method of claim 1 further comprising the step of examining one or more vector displacement maps of skin pores.

4. The method of claim 3 further comprising the step of identifying the principle axis of stretch and or shrink in the vector displacement maps.

5. The method of claim 4 further comprising the step of calculating the strain along the principle axis of stretch and or shrink.

6. The method of claim 3 that uses digital image speckle correlation to generate the vector displacement maps.

7. The method of claim 1 wherein the skin is located on one or more of the face, neck, chest, back, arm, leg, hand or foot.

8. A non-invasive, in-vivo method of characterizing the physical behavior of a skin-molding agent, comprising the steps of:
applying to a portion of skin, a skin-molding agent or a composition comprising a skin-molding agent; and
tracking the displacement of skin pores caused by the application of the skin-molding agent.

9. The method of claim 8 wherein the characterization is one or more quantifications or qualifications, and the physical behavior to be characterized is selected from the group consisting of wetting, dewetting, homogeneity, anisotropy, stretch or shrink along a given direction, drying time and film formation.

10. The method of claim 9 further comprising the step of examining one or more vector displacement maps of skin pores.

11. The method of claim 10 that uses digital image speckle correlation to generate the vector displacement maps.

12. The method of claim 11 further comprising the step of entering the quantifications and/or qualifications into a database.

13. The method of claim 8 wherein the skin is located on one or more of the face, neck, chest, back, arm, leg, hand or foot.

14. The method of claim 8 wherein the application of the skin-molding agent or composition comprising the skin-molding agent is repeated, as part of a defined skin treatment regimen, so that the characterization the skin-molding agent is specific to the defined treatment regimen.

15. A method of developing immediate-effect skin molding compositions comprising the steps of:
using digital image speckle correlation to characterize the behavior of one or more immediate-effect skin molding agents or the behavior of one or more compositions comprising immediate-effect skin molding agents,
comparing the characterized behavior of different skin-molding agents or compositions; and
based on the comparison, adjusting one or more of the skin-molding agents or compositions to improve a particular behavior thereof.

16. The method of claim 15 wherein the improved behavior is selected from the group consisting of wetting, dewetting, homogeneity, anisotropy, stretch or shrink along a given direction, drying time, film formation and physical comfort.

17. A composition made according to the method of claim 15.
